(19) United States
(12) Patent Application Publication
(10) Pub. No.: US 2011/0143366 A1
(43) Pub. Date: Jun. 16, 2011

(54) PROTEIN BIOMARKERS FOR IN VITRO TESTING OF DEVELOPMENTAL TOXICITY AND ENBRYOTOXICITY OF CHEMICAL SUBSTANCES

(75) Inventor: Andre Schrattenholz, Mainz (DE)

(73) Assignee: PROTEOSYS AG, Mainz (DE)

(21) Appl. No.: 12/996,445

(22) PCT Filed: Jun. 4, 2009
(86) PCT No.: PCT/EP09/04016

§ 371 Date: Jan. 18, 2011

Related U.S. Application Data
(60) Provisional application No. 61/129,093, filed on Jun. 4, 2008.

(51) Int. Cl. G01N 33/53; C12Q 1/02

(52) U.S. Cl. 435/7.1; 435/29

(57) ABSTRACT

Presently, the toxicological assessment of chemicals is mainly performed in vivo using a variety of animal species and in addition taking into account human clinical, biochemical, pathological and morphological data. Over the past years it became increasingly clear that some substances are particularly harmful for children and thus there is a focus on the special vulnerability of the developing human brain. Meanwhile there is a recommendation to test substances with a known neurotoxic or teratogenic (in particular a neuroteratogenic) risk additionally for embryotoxicity. Moreover the US Environmental Protection Agency (EPA) requires embryotoxicity tests for pesticides. Further tests are required if substances shall be used as medicaments (S7A Safety Pharmacology Studies for Human Pharmaceuticals, Guidelines of the International Conference on Harmonization, ICH, 2001).

### BACKGROUND OF THE INVENTION

Presently, the toxicological assessment of chemicals is mainly performed in vivo using a variety of animal species and in addition taking into account human clinical, biochemical, pathological and morphological data. Over the past years it became increasingly clear that some substances are particularly harmful for children and thus there is a focus on the special vulnerability of the developing human brain. Meanwhile there is a recommendation to test substances with a known neurotoxic or teratogenic (in particular a neuroteratogenic) risk additionally for embryotoxicity. Moreover the US Environmental Protection Agency (EPA) requires embryotoxicity tests for pesticides. Further tests are required if substances shall be used as medicaments (S7A Safety Pharmacology Studies for Human Pharmaceuticals, Guidelines of the International Conference on Harmonization, ICH, 2001).

The investigation of developmental neurotoxicity of chemicals is regulated by a guideline of the US EPA (test guideline 870.63000) and a draft guideline of the OECD (OECD guideline 426). In vivo studies according to these guidelines include morphological investigations of brains of test animals (usually rats), sets of behavioral tests, investigation of development of young animals (up to adult stage), measurements of biomarkers for gliosis and cytotoxicity and moreover investigations of additional biomarkers. These toxicity tests require a huge number of test animals: About 140 maternal animals and 1000 of their offspring would be consumed over 3-4 months for each substance. Due to the technical and logistic requirements, these in vivo tests are very personnel- and cost intensive. A critical point is, however, that in the corresponding guidelines and animal no reliable and unambiguous end points can clearly be defined. It is uncertain whether the current tests are truly predictive for human development of the central nervous system and related toxicity. In the US, this situation has led to petitions of animal rights and protection organizations like PETA, to withdraw guideline OPPTS 870.6300.

Presently, approximately 100,000 chemical substances are available on the market of the EU. Solid toxicological data, however, are available only for a small percentage of these substances. Especially for chemicals marketed before 1981 there is a lack of safety data. Therefore, risks for employees, consumers and the environment cannot be assessed comprehensively. To improve this unsatisfactory situation the European Commission submitted the so called REACH concept standing for Registration, Evaluation and Authorization of Chemicals. The aim of REACH is to systematically evaluate the risk of the chemical substances produced, used or imported in volumes of more than 1 tonne per year. The burden of proof of the safety of chemicals will be imposed on the manufacturers and fabricators. With regard to the REACH legislation in Europe and similar developments in the US and Japan (Schrattenholz and Klemm, 2006 and 2007) it is likely that the requirements for testing chemicals for developmental neurotoxicity will lead to an enormous increase in the consumption of test animals in the foreseeable future.

On this background, the development of highly predictive, time-efficient in vitro tests for toxicity-related screening is increasingly important. Cell culture models would be positioned as an alternative to highly controversial and problematic, sometimes unsavory animal experiments. The aim is to replace animal tests currently required by legislation for assessment of neurotoxicity and in particular neurodevelopmental toxicity, which are very cost- and time-intensive.

In vitro models have been employed in the field of pharmacological industry for several years. Many of the current in vitro assays involve differentiation models using embryonic stem cells. The embryonic stem cell test (EST) has shown very promising results and the test was able to distinguish strong teratogenes from moderate or non-embryotoxic compounds (Spielmann et al., 1997). The EST takes advantage of the potential of murine embryonic stem cells to differentiate in culture to test embryotoxicity in vitro. This model is limited in part because toxicological end points are defined only for compounds that impair cardiac differentiation.

Thus, there remains a need in the art for an improved in vitro method for reliably determining toxicity of chemicals and pharmaceuticals. In particular, there is a need in the art to provide novel, fast and intelligent in vitro test strategies for developmental toxicity.

It is the object of the present invention, to provide methods and reagents for in vitro screening of toxicity and in particular developmental toxicity of chemical substances.

### DESCRIPTION OF THE INVENTION

The present inventors found out that specific protein biomarkers are diagnostic for developmental toxicity of chemical and pharmaceutical compounds. The impact of a substance on these biomarkers is predictive for the developmental toxicity of the substance. Said impact can be determined by contacting a cell sample wherein at least one of the protein biomarkers is produced, with the substance and determining a variation of said protein biomarker(s) in the cell sample as a result of the exposure to the substance.

In one aspect, the invention provides an in vitro method for the determination of developmental toxicity of a substance, comprising the steps
(i) exposing a cell sample to the substance, and
(ii) detecting a variation of one or more protein biomarkers in the cell sample as a result of the exposure to the substance.

The “protein biomarkers” of the invention are selected from the group consisting of heat shock protein beta-1 (HspB1), Ras-GTPase-activating protein SH3-domain binding protein (G3BP); Ran binding protein 5 (RanBP5), Calreticulin (Calr), Dihydropyrimidinase-like 2 (DRP2), stress-induced phosphoprotein 1 (STIP1), U2af2 protein (U2AF), calcium binding protein 39, isoform CRA_b (Cab39), NmrA-like family domain containing 1 (NMRL1) and post-translational isoforms thereof.

The biomarkers of the invention are well known proteins. The common nomenclature of the proteins is summarized in Table 1:

TABLE 1

| | | | Highly |
|---|---|---|---|
| | | | homologous |
| | Short | | proteins |
| | form | | according to |
| | used in | Synonyms present | BLAST/Expasy |
| Protein name | claims | in the literature | search |
| Heat shock | HspB1 | Heat shock 27 kDa | Alpha-crystallin |
| protein beta-1 | | protein; HSP 27; growth- | B chain |
| | | related 25 kDa protein; | |
| | | P25; HSP25; | |
| Ras-GTPase- | G3BP | Ras-GDP-associated | |
| activating protein | | endoribonuclease; G3BP; | |
| SH3-domain | | G3BP protein; | |
| binding protein | | MKIAA4115 protein | |
| Ran binding | RanBP | Importin subunit beta-3; | HEAT repeat |
| protein 5 | 5 | karyopherin beta-3; Kap | family protein |
| | | beta 3 protein; karybeta3 | |
| Calreticulin | Calr | Calr protein; Crc protein | Calreticulin |
| | | | precursor; |
| | | | Calreticulin-like |
| | | | protein; |
| | | | calreticulin |
| | | | family protein |
| Dihydro- | DRP2 | Ulip2 protein | DRP/CRMP/ |
| pyrimidinase-like | | D-hydantoinase-and | DPYSL proteins |
| 2 | | dihydropyrimidase- | 1 and 3-4 |
| | | related protein, collapsin | |
| | | response mediator | |
| | | protein; | |
| | | Dpysl2-prov protein; | |
| | | Crmp2 protein | |
| Stress-induced | STIP1 | STIP1 protein; sti1-like | Heat shock |
| phosphoprotein 1 | | protein; STI1 | protein 60; |
| | | | Hsc70/Hsp90- |
| | | | organizing |
| | | | protein HOP; |
| | | | TPR domain |
| | | | containing |
| | | | protein |
| U2af2 protein | U2AF | U2 small nuclear RNA | |
| | | auxiliary factor 2; Splicing | |
| | | factor U2AF 65 kDa | |
| | | subunit; U2 small nuclear | |
| | | ribonucleoprotein auxiliary | |
| | | factor; Splicing factor u2af | |
| | | large subunit | |
| Calcium binding | Cab39 | Cab39 protein; MO25-like | |
| protein 39, | | protein; MGC68674 | |
| isoform CRA_b | | protein | |
| NmrA-like family | NMRL1 | NmrA-like protein | |
| domain | | precursor; NmrA family | |
| containing 1 | | protein | |

The term “developmental toxicity” relates to any adverse effects induced during pregnancy, or as a result of parental exposure. In particular, developmental toxicity encompasses embryotoxicity.

A “cell sample” suitable for use in the method of the invention is any sample comprising cells or cell components capable to produce at least one of the above protein biomarkers. The cell sample may e.g. be selected from organs, organ samples, tissues, body fluids, cells, and cell lysates.

The cell sample is preferably of vertebrate origin. Particularly preferred are cell samples of mammalian and in particular human origin.

According to a preferred embodiment, a cell sample comprises stem cells. The stem cells may be omnipotent, pluripotent, multipotent and/or oligopotent stem cells. Particularly preferred are embryonic stem cells. Most preferably the stem cells are human embryonic stem cells (hESC).

In the method of the invention, in step (i) a cell sample is exposed to a substance to be tested for developmental toxicity. Preferably, before contacting the cell sample with the substance to be tested, the baseline value of the one or more biomarkers in the sample is determined. Subsequently, in step (ii), a variation of one or more protein biomarkers in the cell sample as a result of the exposure to the substance is detected.

The detection may comprise qualitative and/or quantitative determination of the one or more protein biomarkers. The biomarkers of the invention are well known proteins, the detection of which is within common knowledge in the art. For example, the detection may be effected by means of an immunological assay or immunoassay. In an immunoassay the presence of one or more protein biomarkers is measured using the reaction of an antibody or antibodies to its antigen. The assay takes advantage of the specific binding of an antibody to its antigen. In the detection of the protein biomarkers of the invention, the biomarkers represent the antigens. Preferably, monoclonal antibodies are used for their detection, as they usually only bind to one site of a particular molecule, and therefore provide a more specific and accurate test, which is less easily confused by the presence of other molecules.

For detecting one or more biomarkers of the invention it is also possible to determine the activity thereof and in particular the variation of the activity upon contacting the cell sample with the substance to be tested.

The quantity of a protein biomarker of the invention can be achieved by a variety of methods known in the art. For example in an immunoassay the antibody for the protein biomarker may be labeled. The label may consist of an enzyme, radioisotope, magnetic label or fluorescent label. Other suitable techniques for the detection of a protein biomarker of the invention include Western Blot and ELISA.

In a preferred embodiment of the invention, the variation of the one or more biomarkers upon contacting the cell sample with the substance to be tested is continuously detected. Examples for continuous assays are spectrophotometric assays, flourimetric assays or chemiluminescence assays. Alternatively, the one or more protein biomarkets are determined discontinuously one or more times after contacting the cell sample with the substance to be tested. For example the cell sample or an extract thereof may be subjected to chromatographic separation such as two or three dimensional gel electrophoresis like SDS-PAGE. The separated proteins may be visualized by means of staining. A molecular analysis of the proteins may be effected e.g. by mass spectroscopy.

According to a preferred aspect of the method of the invention, at least one additional biomarker is determined. The one or more additional biomarkers are preferably markers for general cytotoxicity. It is thus possible to differentiate between developmental toxicity and general toxicity. Exemplary markers which behave independently of substance application but are correlated to EC 50 measurements are: Heart shock protein 8 (HSPS), Stress-induced phosphoprotein 1 (P-Isoform 2), fascin homolog 1 actin bundling protein (Fscn1), Heterologous nuclear ribonuclear ribonucleoprotein A/B isoform 2, and posttranslational isoforms thereof. The common nomenclature of the preferred additional biomarkers is summarized in Table 2:

TABLE 2
Markers for general toxicity

| | Short | | Highly homologous |
|---|---|---|---|
| | form | | proteins according |
| | used in | Synonyms present | to BLAST/Expasy |
| Protein name | claims | in the literature | search |
| Heat shock | HSP8 | Heat shock cognate | |
| protein 8 | | 71 kDa protein; Heat | |
| | | shock 70 kDa protein | |
| | | 8 isoform 1; Hsc70 | |
| | | protein; MGC53952 | |
| | | protein; Heat shock | |
| | | protein 70 HSP70; | |
| | | HSP71; HSC70; | |
| | | HSC71 | |
| Fascin homolog | Fscn1 | Fscn1 protein | Fscn protein 2 and 3 |
| 1, actin bundling | | | |
| protein | | | |
| Heterogeneous | hnRNP | Type A/B hnRNP p38; | Musashi homolog; |
| nuclear | | Type A/B hnRNP p40; | RNA-binding |
| ribonucleo- | | Hnrpab protein; AIF- | protein Musashi |
| protein A/B | | C1; S1 protein C2; | homolog |
| isoform 2 | | Nucleic acid binding | |
| | | factor pRM10; Single | |
| | | stranded D box | |
| | | binding factor | |

A further embodiment of the invention relates to the use of one or more protein biomarkers as defined above as markers for the assessment of developmental toxicity of a substance. The protein biomarkers may be monitored in any known in vivo or in vitro model for toxicity, developmental toxicity or embryotoxicity.

Another embodiment of the invention is a kit for the determination of developmental toxicity of a substance comprising one or more cell samples, wherein preferred cell samples are as defined above. The kit further comprises means for the determination of one or more protein biomarkers. According to a preferred aspect of the invention, the kit further comprises means for determining at least one additional biomarker. The one or more additional biomarkers are preferably markers for general cytotoxicity. Most preferably, the kit comprises means for determining the additional markers Heart shock protein 8 (HSPS), Stress-induced phosphoprotein 1 (P-Isoform 2), fascin homolog 1 actin bundling protein (Fscn1), Heterologous nuclear ribonuclear ribonucleoprotein A/B isoform 2, and/or posttranslational isoforms thereof.

The protein biomarkers of the invention are well known proteins. However, the invention for the first time describes that the specific proteins are diagnostic biomarkers for developmental toxicity of chemical and pharmaceutical compounds.

### EXPERIMENTAL BACKGROUND

The inventors have applied a differential proteomic technology to the quantitative and statistical analysis of protein biomarkers from rodent and human samples related to developmental toxicity. These samples included:
Protein lysates from a variety of experiments carried out for the validation of the EST test in two independent laboratories. Cardiomyocytes differentiated from murine embryonic stem cells according to a standardized protocol (ECVAM validated alternative test) were exposed to sets of substances with known embryotoxic potency and functionally controlled in dose-dependent manner.
Protein lysates from neural cell cultures differentiated from murine embryonic stem cells after exposure to known embryotoxic substances.
Protein lysates from neural cell cultures differentiated from human embryonic stem cells after exposure to known embryotoxic substances.

High quality lysates from neurally differentiated human embryonic stem cell have been submitted to this type differential proteomic analysis. The hESC cultures have been treated with methyl mercury and valproic acid. Samples (including treated and non-treated undifferentiated hESC and respective neural precursors) have been radiolabelled and submitted to a differential quantitative pattern analysis using high resolution 2D-PAGE as described previously (e.g. Schrattenholz & Groebe 2007; Groebe et al., 2007; Wozny et al., 2007): 177 protein spots have been found to be differentially affected by the treatment, among them many redundant posttranslational isoforms, have been identified so far using automated high-throughput MALDI-TOF mass spectrometry. Among proteins identified, there are nuclear, cytoskeletal, extracellular matrix and stress proteins, and proteins involved in protein turnover. The significance of these findings has to be seen in the context of corresponding results obtained from material from mESC (cardiomyocytes, EST-test and mESC neurons) and will be discussed below.

In a similar way, lysates from MgHgCl-treated mESC differentiated to neural cells by partner and lysates from mESC differentiated to cardiomyoctes (material from the enlargement of the database of the validated EST-test) obtained after substance-treatment in different laboratories have been investigated. For the mESC neural cells, 93 differential spots were found and identified. The biological significance of the corresponding biomarker signature will be discussed below in the context of further but similar and closely related data from cardiomyocytes.

The biggest data set was obtained using the lysates from substance testing in the EST model at two independent laboratories and applying a pooling scheme previously successfully tested and published. The key of this strategy is quantitative and statistically reliable control of complex patterns of proteins spots and/or peaks after analysis of complex biological samples by 2D-PAGE or multidimensional LC (Groebe et al., 2007, \check{S}o\check{s}kić et al., 2008).

Substances tested at the two sites included Dinoseb, Nitrofen, Ochratoxin-A, Lovastatin, MAM, β-aminoproprionitril, Metoclopramide, Doxylamine, D-Penicillamine, Pravastatin, Warfarin and Furosemide. Across the individual differential analyses for each of substance treated EST lysates, 380 differential proteins were found and identified by automated high-throughput MALDI-TOF mass spectrometry. There was a substantial number of redundant protein isoforms pointing to extensive posttranslational modifications. The differential quantitative data were submitted to a cluster analysis (shown in FIG. 1 below) which revealed three clusters, assorting the substances in a very meaningful way: cluster 1 comprising mainly highly embryotoxic, cluster 2 with non-embryotoxic and cluster 3 rather with moderately embryotoxic substances. It is noteworthy that although the biological side was only controlled in terms of IC50 values, but not in terms of numbers, activity amplitudes and percentages of cell types, i.e. had a huge degree of heterogeneity and stochasticity, the wealth of molecular data nevertheless reveals the following:
1. The molecular signatures are able to assort substance effects.
2. They also help to indicate failed or highly aberrant experiments.
3. Only about 15-20 protein biomarkers behave in a significant way and representatively for all substances.
4. Some of these and interestingly mainly cytoskeletal proteins show a uniform behaviour for all conditions, independent of substance or cluster: We interpret these as more likely to be representative for general cytotoxicity or cell stress.
5. But some protein biomarkers, present in several redundant isoforms clearly behave in a graded fashion depending on supposed embryo toxicity of substances. These include regulatory elements of ras pathway and small GTPases as well as regulatory elements of the calcium-dependent IP3 pathway. These pathways and proteins have well established roles in embryogenesis and are extremely plausible in the context of embryo toxicity.
6. The ongoing bioinformatic effort and data mining shows that these few (>10) biomarker candidates have the potential of being true markers for embryotoxicity.

There is a partial overlap of these signatures with the proteins identified from hESC and mESC derived neurons treated with MgHgCl and valproic acid which points to a general significance of the underlying markers for general embryotoxicity.

The determined protein biomarkers for embryotoxicity are shown in Table 3.

TABLE 3
Proteins biomarkers for embryotoxicity

| | Gene bank | | | |
|---|---|---|---|---|
| | accession # | | | |
| | for mouse | | | |
| Protein name | homologue | Cluster 1 | Cluster 2 | Cluster 3 |
| Heat shock protein | gi\|547679 | down | up | up |
| beta-1 (HspB1) (Heat | gi\|7305173 | | | |
| shock 27 kDa protein) | | | | |
| (HSP 27) (Growth- | | | | |
| related 25 kDa | | | | |
| protein) (P25) | | | | |
| (HSP25) | | | | |
| Ras-GTPase- | gi\|7305075 | up | down | up |
| activating protein SH3- | | | | |
| domain binding | | | | |
| protein | | | | |
| Ras-GDP-associated | | | | |
| endoribonuclease | | | | |
| G3BP | | | | |
| Ran binding protein 5 | gi\|12057236 | down | up | down |
| | gi\|29789199 | | | |
| | gi\|148668272 | | | |
| Calreticulin | gi\|6680836 | up | no | up |
| | | | change | |
| Unnamed protein | gi\|74200069 | up | no | up |
| product | | | change | |
| (calreticulin family) | | | | |
| Dihydropyrimidinase- | gi\|40254595 | down | down | up |
| like 2 (Ulip2 protein) | gi\|1915913 | | | |
| D-hydantoinases and | | | | |
| dihydropyrimidase- | | | | |
| related proteins, | | | | |
| collapsin response | | | | |
| mediator proteins | | | | |
| Stress-induced | gi\|13277819 | down | up | no |
| phosphoprotein 1 | | | | change |
| (P-isoform 1) | | | | |
| U2af2 protein | gi\|63101571 | no | down | no |
| | | change | | change |
| Calcium binding | gi\|148708308 | up | no | no |
| protein 39, isoform | gi\|18044843 | | change | change |
| CRA_b | | | | |
| NmrA-like family | gi\|24431937 | down | up | down |
| domain containing 1 | | | | |

Cluster 1 shows the alterations of corresponding marker proteins after treatment of the EST model with highly embryotoxic substances Dinoseb, Ochratoxin, Nitrofen, Lovastatin; Cluster 2 shows the situation when non-embryotoxic substances were used in this model (β-aminoproprionitril, metoclopramide, doxylamine, D-penicillamine) and cluster 3 the effects of application of moderately embryotoxic substances like pravastatin and furosemide. The combination of these markers will allow to discriminate in vitro embryotoxic properties of substances.

Markers which behave independently of substance application but are correlated to EC 50 measurements in the EST model rather represent general cytotoxicity are shown in Table 4:

TABLE 4

| | Gene bank | | | |
|---|---|---|---|---|
| | accession # | | | |
| | for mouse | | | |
| Protein name | homologue | Cluster 1 | Cluster 2 | Cluster 3 |
| Heat shock protein 8 | gi\|42542422 | down | down | down |
| Stress-induced | gi\|13277819 | down | down | down |
| phosphoprotein 1 | | | | |
| (P-Isoform 2) | | | | |
| Fscn1 protein, fascin | gi\|144719132 | down | down | down |
| homolog 1, actin | gi\|113680348 | | | |
| bundling protein | | | | |
| Heterogeneous | gi\|6754222 | up | up | up |
| nuclear | gi\|26345118 | | | |
| ribonucleoprotein | gi\|12851175 | | | |
| A/B isoform 2 | | | | |

The relevant literature to these proteins can be accessed using the Gene bank accession numbers in the tables. In particular the Ras-GTPase-activating protein SH3-domain binding protein (G3BP), the dihydropyrimidinase-related protein2 (DRP2) and the Ran binding protein 5 (RanBP5) have reported roles in development, neurodevelopment and embryogenesis: For G3BP a crucial role in fetal growth and embryogenesis has been shown (Zekri et al., 2005; Lypowy et al., 2005), as involvement in important oncogenic pathways as e.g. the p53 tumor suppressor pathway, a critical step in human tumorigenesis (Kim et al., 2007). Receptor tyrosine Kinase (RTK)/Ras GTPase/MAP kinase (MAPK) signaling pathways are used ubiquitously during development to control many different biological processes. Small GTPases of the Ras superfamily are key regulators of diverse cellular and developmental events, including differentiation, cell division, vesicle transport, nuclear assembly, and control of the cytoskeleton during differentiation (some recent reviews: Omerovic et al., 2007; Wodarz and Näthke, 2007; Kratz et al., 2007).

In the case of RanBP5 the same is true, because Ran as well is a member of the Ras superfamily of small GTPases (Lundquist 2006) treated above.

RanBP=karyopherin or transportin imports numerous RNA binding proteins into the nucleus binding substrates in the cytoplasm and targeting them through the nuclear pore complex, where RanGTP dissociates them in the nucleus (e.g. Cansizoglu and Chook 2007). Again a role on differentiation, development and carcinogenesis is apparent (Teng et al., 2007).

Originally the four members of the DRP-gene family identified in humans were found being expressed mainly in fetal and neonatal brains of mammals and chickens, and have been implicated as intracellular signal transducers in the development of the nervous system (Kitamura et al., 1999; Arimura et al., 2004; Schmidt and Strittmatter, 2007;). DRP-2 has been reported to contribute to the pathfinding of growing axons during brain development (Weitzdoerfer et al., 2001; Inagaki et al., 2000). DRP2 has also been shown to play role in the response to neuronal stress (e.g. Sommer et al., 2004; Butterfield et al., 2006).

Interestingly also for HspB1 a key role in differentiation of trophoblast cells, which is a critical process for the proper establishment of the placenta and therefore necessary to maintain embryonic development, has been reported recently (Winger et al., 2007). HspB1 is part of the mitogen-activated protein kinase (MAPK) pathways mediating some important cellular processes likely regulating preimplantation development (Natale et al., 2004).

Taken together the role of the proteins found in embryogenesis and neonatal development is very plausible and the detailed molecular information revealed by the present application will help to predict the impact of potentially embryotoxic substances in vitro.

### FIGURES

FIG. 1 shows a cluster analysis of proteins differentially affected by substance treatment in the EST model. Red indicates up, and green down regulation of expression in the protein lysates. There are only a few proteins which clearly behave in a substance- and cluster-dependent way across all conditions; these are promising candidates for markers of embryo toxicity.

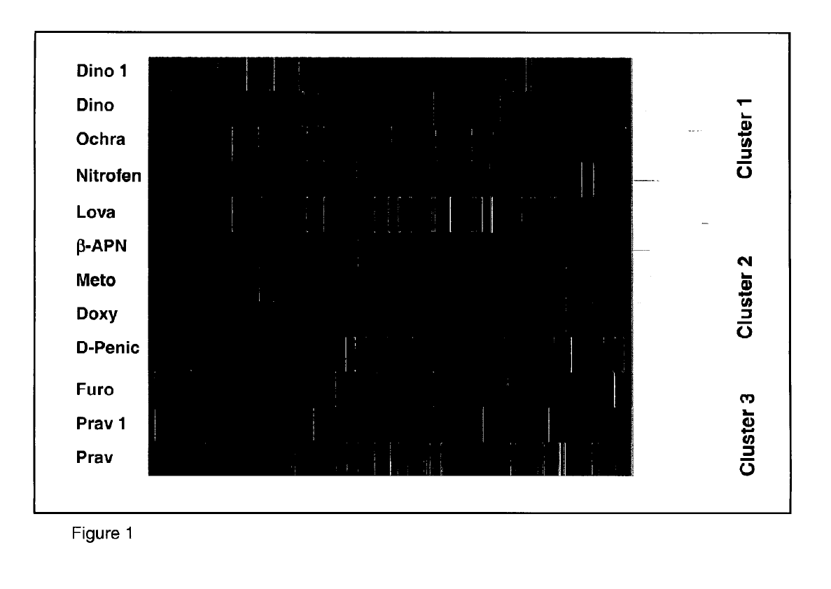

### REFERENCES

Arimura N, Menager C, Fukata Y, Kaibuchi K. Role of CRMP-2 in neuronal polarity. J. Neurobiol. 2004 January; 58(1):34-47.
Balls, M.; Goldberg, A. M.; Fentem, J. H.; Broadhead, C. L.; Burch, R. L.; Festing, M. F.; Frazier, J. M.; Hendriksen, C. F.; Jennings, M.; van der Kamp M. D.; Morton, D. B.; Rowan, A. N.; Russell, C.; Russell, W. M.; Spielmann, H.; Stephens, M. L.; Stokes, W. S.; Straughan, D. W.; Yager, J. D.; Zurlo, J.; van Zutphen, B. F. (1995) The three Rs: the way forward: the report and recommendations of ECVAM Workshop 11 ATLA 23, 838-866.
Butterfield D A, Perluigi M, Sultana R. Oxidative stress in Alzheimer's disease brain: new insights from redox proteomics. Eur J. Pharmacol. 2006 Sep. 1; 545(1):39-50. Epub 2006 Jun. 15.
Cansizoglu A E, Chook Y M. Conformational heterogeneity of karyopherin beta2 is segmental. Structure. 2007 November; 15(11):1431-41.
Genschow, E., Spielmann, H., Scholz, G., Pohl, I., Seiler, A., Cleman, N., Bremer, S. and Becker, K. (2004). Validation of the embryonic stem cell test in the international ECVAM validation study on three in vitro embryotoxicity tests. ATLA 32, 209-244.
Genschow, E., Spielmann, H., Scholz, G., Seiler, A., Brown, N., Piersma, A., Brady, M., Huuskonen, H., Paillard, F., Bremer S., Clemann, N. and Becker, K. (2002) The ECVAM international validation study on in vitro embryotoxicity tests. Results of the definitive phase and evaluation of prediction models. ATLA, 30, 151-176.
Groebe K, Krause F, Kunstmann B, Unterluggauer H, Sastri C, Stegmann W, Wozny W, Schwall G P, PoznanovićS, Dencher N A, Jansen-Dũrr P, Osiewacz H D and Schrattenholz A (2007) Differential proteomic profiling of mitochondrial preparations from Podospora anserina, rat and human reveals distinct patterns of age-related oxidative changes, Exp. Gerontology, 42, 887-898.
Inagaki H, Kato Y, Hamajima N, Nonaka M, Sasaki M, Eimoto T. Differential expression of dihydropyrimidinase-related protein genes in developing and adult enteric nervous system. Histochem Cell Biol. 2000 January; 113(1):37-41.
Kim M M, Wiederschain D, Kennedy D, Hansen E, Yuan Z M. Modulation of p53 and MDM2 activity by novel interaction with Ras-GAP binding proteins (G3BP). Oncogene. 2007 Jun. 21; 26(29):4209-15. Epub 2007 Feb. 5.
Kitamura K, Takayama M, Hamajima N, Nakanishi M, Sasaki M, Endo Y, Takemoto T, Kimura H, Iwaki M, Nonaka M. Characterization of the human dihydropyrimidinase-related protein 2 (DRP-2) gene. DNA Res. 1999 Oct. 29; 6(5):291-7.
Kratz C P, Niemeyer C M, Zenker M. An unexpected new role of mutant Ras: perturbation of human embryonic development. J Mol Med. 2007 March; 85(3): 227-35. Epub 2007 Jan. 9.
Lundquist E A. Small GTPases. WormBook. 2006 Jan. 17; 1-18.
Lypowy J, Chen I Y, Abdellatif M An alliance between Ras GTPase-activating protein, filamin C, and RasGTPase-activating protein SH3 domain-binding protein regulates myocyte growth. J Biol Chem. 2005 Jul. 8; 280(27): 25717-28. Epub 2005 May 9.
Natale D R, Paliga A J, Beier F, D'Souza S J, Watson A J. p38 MAPK signaling during murine preimplantation development. Dev Biol. 2004 Apr. 1; 268(1): 76-88.
Omerovic J, Laude A J, Prior I A. Ras proteins: paradigms for compartmentalised and isoform-specific signalling. Cell Mol Life Sci. 2007 October; 64(19-20):2575-89.
Piersma. A. H., Genschow, E., Verhoef, A., Spanjersberg, M. Q. I., Brown, N. A., Brady, M., Burns, A., Clemann, N., Seiler, A., Spielmann, H. (2004). Validation of the postimplantation rat whole embryo culture test in the international ECVAM validation study on three in vitro embryotoxicity tests. ATLA 32, 275-307.
Schmidt E F, Strittmatter S M. The CRMP family of proteins and their role in Sema3A signaling. Adv Exp Med Biol. 2007; 600:1-11.
Schrattenholz A and Groebe K (2007) What does it need to be a biomarker? Relationships between resolution, differential quantification and statistical validation of protein surrogate biomarkers. Electrophoresis, 28(12), 1970-1979.
Schrattenholz A. and Klemm M. How Human Embryonic Stem Cell Research Can Impact In Vitro Drug Screening Technologies of the Future. In: Drug Testing in vitro by Marx U. and Sandig V. (eds.), 2006 Wiley-VCH, Weinheim 205-228.
Schrattenholz A and Klemm M (2007) Neuronal Cell Culture from Human Embryonic Stem Cells as in vitro Model for Neuroprotection. ALTEX, 24(1), 9-15.
Seiler, A., Buesen, R., Visan, A., and Spielmann, H. (2005). Use of Murine Embryonic Stem Cells in Embryotoxicity Assays: The Embryonic Stem Cell Test. In: Methods in Molecular Biology: Embryonic Stem Cells-II, Edited by: K. Turksen, Humana Press, Totowa, N.J., USA. in press.
Seiler, A., Visan, A., Buesen, R., Slawik, B., Genschow, E., and Spielmann H. (2004). Improvement of an in vitro stem cell assay (EST) for developmental toxicity by establishing molecular endpoints of tissue-specific development. Reproductive Toxicology 18: 231-240.
Sommer S, Hunzinger C, Schillo S, Klemm M, Biefang-Arndt K, Schwall G, Pütter S, Hoelzer K, Schroer K, Stegmann W Schrattenholz A (2004) Molecular analysis of homocysteic acid-induced neuronal stress. Journal of Proteome Research 3(3), 572-581.
\check{S}o\check{s}ić kid V, Groebe K and Schrattenholz A (2008). Nonenzymatic posttranslational modifications in ageing. Exp. Gerontology 43(4), in press.
Teng S C, Wu K J, Tseng S F, Wong C W, Kao L. Importin KPNA2, NBS1, DNA repair and tumorigenesis. J Mol Histol. 2006 September; 37(5-7):293-9. Epub 2006 Jun. 3.
Weitzdoerfer R, Fountoulakis M, Lubec G. Aberrant expression of dihydropyrimidinase related proteins-2,-3 and -4 in fetal Down syndrome brain. J Neural Transm Suppl. 2001; (61):95-107.
Winger Q A, Guttormsen J, Gavin H, Bhushan F Heat shock protein 1 and the mitogen-activated protein kinase 14 pathway are important for mouse trophoblast stem cell differentiation. Biol Reprod. 2007 May; 76(5):884-91. Epub 2007 Jan. 31.
Wodarz A, Näthke I. Cell polarity in development and cancer. Nat Cell Biol. 2007 September; 9(9):1016-24.
Wozny W, Schroer K, Schwall G P, Poznanović S, Stegmann W, Dietz K, Rogatsch H, Schaefer G, Huebl H, Klocker H, Schrattenholz A, Cahill M A (2007) Differential radioactive quantification of protein abundance ratios between benign and malignant prostate tissues: cancer association of annexin A3. Proteomics, 7(2), 313-322.
Zekri L, Chebli K, Tourriére H, Nielsen F C, Hansen T V, Rami A, Tazi J. Control of fetal growth and neonatal survival by the RasGAP-associated endoribonuclease G3BP. Mol Cell Biol. 2005 October; 25(19):8703-16.

1. An in vitro method for the determination of developmental toxicity of a substance, comprising the steps
(i) exposing a cell sample to the substance, and
(ii) detecting a variation of one or more protein biomarkers in the cell sample as a result of the exposure to the substance,
wherein the protein biomarkers are selected from the group consisting of heat shock protein beta-1 (HspB1), Ras-GTPase-activating protein SH3-domain binding protein (G3BP), Ran binding protein 5 (RanBP5), Calreticulin (Calr), Dihydropyrimidinase-like 2 (DRP2), stress-induced phosphoprotein 1 (STIP1), U2af2 protein (U2AF), calcium binding protein 39, isoform CRA_b (Cab39), NmrA-like family domain containing 1 (NMRL1), and post-translational isoforms thereof.

2. The method of claim 1 wherein the cell sample is selected from the group consisting of organ samples, tissues, body fluids, cells, and cell lysates.

3. The method of claim 1, wherein the cell sample comprises vertebrate cells, in particular mammalian cells such as human cells.

4. The method of claim 1, wherein the cell sample comprises stem cells, in particular omnipotent, pluripotent, multipotent and/or oligopotent stem cells.

5. The method of claim 1, for the determination of embryotoxicity, wherein the cell sample comprises embryonic stem cells.

6. The method of claim 1, wherein step (ii) comprises the qualitative or quantitative determination of the one or more biomarkers.

7. The method of claim 1, wherein the variation of one or more protein biomarkers in the cell sample is determined continuously.

8. The method of claim 1, wherein the determination of the one or more biomarkers comprises an immunological assay, activity assay, and/or molecular assay.

9. The method of claim 1, wherein the determination of the one or more biomarkers comprises fluorescence detection.

10. The method of claim 1, further comprising the determination of at least one additional protein biomarker selected from the group comprising Heart shock protein 8 (HSP8), Stress-induced phosphoprotein 1 (P-Isoform 2), fascin homolog 1 actin bundling protein (Fscn1), and Heterologous nuclear ribonuclear ribonucleoprotein A/B isoform 2, and post-translational isoforms thereof.

11. The use of one or more proteins selected from the group consisting of heat shock protein beta-1 (HspB1), Ras-GTPase-activating protein SH3-domain binding protein (G3BP), Ran binding protein 5 (RanBP5), Calreticulin (Calr), Dihydropyrimidinase-like 2 (DRP2), stress-induced phosphoprotein 1 (STIP1), U2af2 protein (U2AF), calcium binding protein 39, isoform CRA_b (Cab39), NmrA-like family domain containing 1 (NMRL1), heat shock protein 8 (HSP8), fascin homolog 1, acting bundling protein (Fscn1), heterogeneous nuclear ribonucleoprotein NB isoform 2 (hnRNP) as biomarkers for the determination of developmental toxicity of a substance.

12. The use of claim 11 for the determination of embryotoxicity.

13. A kit for the determination of developmental toxicity of a substance comprising
one or more cell samples, and
means for the determination of one or more protein biomarkers selected from the group consisting of heat shock protein beta-1 (HspB1), Ras-GTPase-activating protein SH3-domain binding protein (G3BP), Ran binding protein 5 (RanBP5), Calreticulin (Calr), Dihydropyrimidinase-like 2 (DRP2), stress-induced phosphoprotein 1 (STIP1), U2af2 protein (U2AF), calcium binding protein 39, isoform CRA_b (Cab39), NmrA-like family domain containing 1 (NMRL1) and post-translational isoforms thereof, heat shock protein 8 (HSP8), fascin homolog 1, acting bundling protein (Fscn1), heterogeneous nuclear ribonucleoprotein NB isoform 2 (hnRNP).

14. The kit of claim 13, wherein the cell sample comprises embryonic stem cells, in particular human embryonic stem cells.

15. The kit of claim 13, further comprising means for determining at least one additional protein biomarker selected from the group consisting of heat shock protein 8 (HSP8), fascin homolog 1, acting bundling protein (Fscn1), heterogeneous nuclear ribonucleoprotein NB isoform 2 (hnRNP).
